(19) United States
(12) Patent Application Publication
(10) Pub. No.: US 2010/0324138 A1
(43) Pub. Date: Dec. 23, 2010

(54) Lipoxin A4 Protection for Retinal Cells

(76) Inventor: Nicolas G. Bazan, New Orleans, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 12/740,286

(22) PCT Filed: Oct. 29, 2008
(86) PCT No.: PCT/US08/81521

§ 371 Date: Jul. 23, 2010

Related U.S. Application Data
(60) Provisional application No. 60/983,447, filed on Oct. 29, 2007.

(51) Int. Cl. A61K 31/202; A61P 27/02

(52) U.S. Cl. 514/560

(57) ABSTRACT

Lipoxin A4 and its analogs have been found to be effective in inhibiting apoptosis of retinal pigment epithelial cells induced by oxidative stress. Thus lipoxin A4 and its analogs, for example, lipoxin A4 epimer 15, can be used to prevent and treat retinal diseases due to the progressive degeneration of photoreceptors and retinal pigment epithelial cells (RPE cells), e.g., the dry form of age-related macula degeneration. They can also be combined with other compounds known to prevent apoptosis in retinal pigment epithelial cells, e.g., docosahexaenoic acid and neuroprotectin D1.

The benefit of the filing date of provisional U.S. application Ser. No. 60/983,447, filed Oct. 29, 2007, is claimed under 35 U.S.C. §119(e) in the United States, and is claimed under applicable treaties and conventions in all countries.

The development of this invention was partially funded by the Government under grant number EY05121 from the National Institutes of Health National Eye Institute, and grant number P20 RR016816 from the National Institutes of Health National Center for Research Resources. The Government has certain rights in this invention.

### TECHNICAL FIELD

This invention involves the use of lipoxin A4 or its analogs to prevent and treat retinal diseases due to the progressive degeneration of photoreceptors and retinal pigment epithelial cells (RPE cells), e.g., the dry form of age-related macula degeneration.

### BACKGROUND ART

### Retinal Pigment Epithelial Cells And Retinal Diseases

Photoreceptor outer segments contain rhodopsin as well as the highest content of docosahexaenoic acid (DHA) of any cell type. In contact with the photoreceptor tips is a monolayer of cells, the retinal pigment epithelium (RPE), derived from neuroepithelium. These cells are the most active phagocytes of the body. In a daily cycle, they engulf and phagocytize the distal tips of photoreceptor outer segments, thereby participating in rod outer segment renewal in a process that is balanced by addition of new membrane to the base of the outer segments. The conservation of DHA in photoreceptors is supported by retrieval through the interphotoreceptor matrix, which supplies the fatty acid for the biogenesis of outer segments. See, Stinson, A. M., Wiegand, R. D. & Anderson, R. E. (1991) J. Lipid Res. 32: 2009-2017; Bazan, N. G., Birkle, D. L. & Reddy, T. S. (1985) in Retinal Degeneration: Experimental and Clinical Studies. Eds. LaVail, M. M., Anderson, R. E., & Hollyfield, J. (Alan R. Liss, Inc., New York) pp. 159-187; and Gordon, W. C., Rodriguez de Turco, E. B. & Bazan, N. G. (1992) Curr. Eye Res. 11: 73-83. The continuous renewal of photoreceptors is tightly regulated so that their length and chemical composition, including that of their phospholipids, are maintained. Photoreceptor phospholipids contain most of the DHA placed at carbon 2 of the glycerol backbone. However, they may also display molecular species of phospholipids containing DHA in both C1 and C2 positions of the glycerol backbone, as well as polyunsaturated fatty acids of longer chains than C22 that result from subsequent elongation of DHA. See, Choe, H-G & Anderson, R. E. (1990) Exp. Eye Res. 51: 159-165. Retina, as well as brain, displays an unusual DHA retention ability, even during very prolonged dietary deprivation of essential fatty acids of the omega-3 family. In fact, to effectively reduce the content of DHA in retina and brain in rodents and even in non-human primates, dietary deprivation for more than one generation has been necessary. Under these conditions, impairments of retinal function have been reported. See, Wheeler, T. G., Benolken, R. M. & Anderson, R. E. (1975) Science. 188: 1312-1314; and Neuringer, M., Connor, W. E., Van Petten, C. & Barstad, L. (1984) J. Clin. Invest. 73: 272-276.

RPE cells also perform several other functions, including transport and re-isomerization of bleached visual pigments, and contribute to the maintenance of the integrity of the blood-outer retinal barrier. Retinal detachment or trauma triggers dysfunctions in the RPE cells that lead to the onset and development of proliferative vitreoretinopathy.

RPE cells are essential for photoreceptor cell survival. When RPE cells are damaged or die, photoreceptor function is impaired, and the photoreceptor cells die as a consequence. Thus, oxidative stress-mediated injury and cell death in RPE cells impair vision, particularly when the RPE cells of the macula are affected. The macula is the area of the retina responsible for visual acuity. The pathophysiology of many retinal degenerations (e.g., age-related macular degenerations and Stargardt's disease) involves oxidative stress leading to apoptosis of RPE cells. In fact, RPE cell damage and apoptosis seem to be the dominant factors in age-related macular degeneration. See, Hinton, D. R., He, S. & Lopez, P. F. (1998) Arch. Ophthalmol. 116:203-209. In Stargardt's disease, the lipofuscin fluorophore A2E mediates RPE damage. The caspase-3 enzyme has been shown to be part of this cascade, whereas the anti-apoptotic Bcl-2 protein exerts cellular protection. See, Sparrow, J. R. Vollmer-Snarr, H. R., Zhou, J., Jang, Y. P., Jockusch, S., Itagaki, Y. & Nakanishi, K. (2003) J. Biol. Chem. 278:18207-18213.

Photoreceptor cells (rods and cones) are highly specialized and differentiated neurons with stacks of photosensitive membrane discs that contain rhodopsin as well as numerous other, far less abundant proteins in their outer segments. Damage to and apoptotic death of photoreceptor cells are hallmarks of retinal degenerative diseases. In retinitis pigmentosa (“RP”), a heterogeneous group of inherited blinding diseases, death of rod photoreceptors initially occurs in the periphery of the retina. In the dry form of age-related macular degeneration (“AMD”), the leading cause of loss of sight in patients over the age of 65, progressive perturbation and loss of visual acuity are caused by photoreceptor death in the center of the retina, the macula. See, Papermaster, D. S. (2002) Invest. Ophthalmol. Vis. Sci., 43: 1300-1309; Rattner A, Nathans J. (2006) Nat. Rev. Neurosci. 7: 860-872; Chang G. Q., Hao Y, Wong F. (1993) Neuron. 11: 595-605; Portera-Cailliau C., Sung C. H., Nathans J., Adler R. (1994) Proc. Natl. Acad. Sci. USA. 91: 974-978; Bird A. C. (2003) Eye. 17: 457-466.

The photoreceptors and RPE cells are constantly subjected to a plethora of environmental as well as intrinsic factors that are potential disruptors of homeostasis, for example, high oxygen tension and intense light during the day. The cell membranes of photoreceptors and RPE cells contain the highest content of all other tissues of polyunsaturated fatty acyl chains in their phospholipids (particularly docosahexaenoic acid (DHA), as well as arachidonic acid (20:4, n-6)). In experimental models of retinal degeneration, lipid peroxidation, a potentially cell-damaging event, occurs in the outer segment discs. See, Organisciak D. T., Darrow R. M., Jiang Y. L., Blanks J. C. (1996) Invest. Ophthalmol. Vis. Sci. 37: 2243-2257. Moreover, in the deposits of debris-like material (also called “drusen”) that accumulate between the RPE cells and Bruch's membrane from patients with AMD, DHA oxidation products have been found to form protein adducts. See, Crabb J. W., Miyagi M., Gu X, et al. (2002) Proc. Natl. Acad. Sci. USA. 99: 14682-14687. Trauma or retinal detachment induces RPE dysfunctions that, in turn, contribute to the onset and development of the proliferation of membrane-like structures and neovascularization. Neovascularization is a cause of the wet form of AMD. RPE cells have developed endogenous mechanisms to cope with these challenges and guard against damage, such as the presence of antioxidants (e.g., vitamin E), which contribute to the preservation of cellular integrity.

### Lipoxins

Lipoxins are biosynthesized from arachidonic acid. See, Bazan N. G. (2006) In Basic Neurochemistry: Molecular, Cellular and Medical Aspects, 7th edition, G. Siegel, R. W. Albers, S. T. Brady, D. L. Price (eds.), Chapter 33: 575-591; and Mattson M. P., Bazan N. G. (2006) In Basic Neurochemistry: Molecular, Cellular and Medical Aspects, 7th edition, G. Siegel, R. W. Albers, S. T. Brady, D. L. Price (eds.), Chapter 35: 603-615. Lipoxins are potent mediators of the resolution phase of the inflammatory response and of dysfunctional immunity. See, Serhan C. N., Takano T., Clish C. B., Gronert K., Petasis N. (1999) Adv. Exp. Med. Biol. 469: 287-293; and Fiorucci S., Wallace J. L., Mencarelli A., et al. (2004) Proc. Natl. Acad. Sci. USA. 101: 15736-15741. Lipoxin A4 and its analogs, including lipoxin A4 epimer 15 (or 15-epi-lipoxin A4), are well known in the art. See, U.S. Pat. Nos. 6,831,186 and 6,645,978; I. M. Fierro et al., “Lipoxin A4 and aspirin-triggered 15-epi-lipoxin A4 inhibit human neutrophil migration: Comparisons between synthetic 15 epimers in chemotaxis and transmigration with microvessel endothelial cells and epithelial cells,” Journal of Immunology, vol. 170, pp. 2688-2694 (2003); G. Bannenberg et al., “Lipoxins and novel 15-epi-lipoxin analogs display potent anti-inflammatory actions after oral administration,” Brit. J. Pharma. Vol. 143, pp. 43-52 (2004); and R. Scalia et al., “Lipoxin A4 stable analogs inhibit leukocyte rolling and adherence in the rat mesenteric microvasculature: role of P-selectin,” Proc. Natl. Acad. Sci. USA. vol. 94, pp. 9967-9972 (1997). Lipoxin A4 and docosahexaenoic acid-derived neuroprotectin D1 (NPD1) are lipid autacoids formed by 12/15 lipoxygenase (LOX) pathways that exhibit anti-inflammatory and neuroprotective properties. Mouse corneal epithelial cells were found to generate both endogenous lipoxin A4 and NPD1. See, K. Gronert et al., A role for the mouse 12/15-lipoxygenase pathway in promoting epithelial wound healing and host defense,” PNAS, vol. 280, pp. 15267-15278 (2005). Lipoxins have been reported to play a role in wound healing in the corneal of the eye. See, K. Gronert, “Lipoxins in the eye and their role in wound healing,” Prostaglandins, Leukotrienes and Essential Fatty Acids, vol. 73, pp. 221-229 (2005). Lipoxin A4 was shown to be formed in healthy and injured corneas, and lipoxygenase (LOX) enzyme activity has been indicated in the cornea of rats and rabbits. In the mouse cornea, lipoxin A4 was found to be generated in the absence of inflammation. In other tissues, lipoxins are predominantly formed during the resolution phase of acute inflammation. Lipoxin A4 or LOX have not been reported from any cells of the back of the eye, only from the corneal epithelial cells. Specifically, neither has been reported from either photoreceptors or retinal pigment epithelial cells. See, also, Bazan, N. et al., “Signal Transduction and Gene Expression in the Eye: A Contemporary View of the Pro-inflammatory, Anti-inflammatory and Modulatory Roles of Prostaglandins and Other Bioactive Lipids,” Survey of Opth., Vol. 41, Supp.2, pp. S23-S34 (1997); Bazan, N. et al., “Arachidonic Acid Cascade and Platelet-Activating Factor in the Network of Eye Inflammatory Mediators: Therapeutic Implications In Uveitis,” Int'l Opth., Vol. 14, pp. 335-344 (1990); and Bazan, N., “Metabolism of Arachidonic Acid in the Retina and Retinal Pigment Epithelium: Biological Effects of Oxygenated Metabolites of Arachidonic Acid,” The Ocular Effects of Prostaglandins and Other Eicosanoids, Pub. Alan R. Liss, Inc., pp. 15-37 (1989).

Lipoxin A4 and its analogs have been proposed as a treatment for dry eye, known generically as keratoconjunctivitis sicca and characterized by lack of moisture or lubrication in the eye. See, U.S. Pat. No. 6,645,978; and U.S. Patent Application Pub. No. U.S. 2005/0255144. Dry eye is known to be a separate condition from the dry form of AMD, which is a disease of the back of the eye that involves the death of photoreceptors and RPE cells.

### DISCLOSURE OF INVENTION

I have discovered that lipoxin A4 and its analogs (e.g., lipoxin A4 epimer 15; also known as 15-epi-lipoxin A4 or 15-epimer lipoxin A4) are very effective in inhibiting apoptosis of retinal pigment epithelial cells induced by oxidative stress. Thus lipoxin A4and its analogs can be used to prevent and treat retinal diseases due to the progressive degeneration of photoreceptors and retinal pigment epithelial cells (RPE cells), e.g., the dry form of age-related macula degeneration.

### BRIEF DESCRIPTION OF DRAWINGS

FIG. 1 illustrates the percent apoptosis in human retinal pigment epithelial cells under either no stress or under oxidative stress (OS) and with various concentrations (nM) of lipoxin A4 (LXA4) or lipoxin A4 epimer 15 (LXA4-epimer).

FIG. 2 illustrates the percent apoptosis in human retinal pigment epithelial cells under either no stress or under oxidative stress (OS) and with various concentrations (nM) of lipoxin A4 (LXA4) or lipoxin A4 epimer 15 (LXA4-epimer) and with various concentrations (nM) of neuroprotectin D1 (NPD1).

FIG. 3 illustrates the amount of induced activation of COX-1 measured as luciferase activity in human retinal pigment epithelial cells exposed to interleukin-1β(IL-1β) and to various concentrations (nM) of lipoxin A4 (LXA4) or lipoxin A4 epimer 15 (LXA4-epimer).

### MODES FOR CARRYING OUT THE INVENTION

### Example 1

### Lipoxin A4 And Lipoxin A4 Epimer 15 Mediated Inhibition f Apoptosis Induced By Oxidative Stress In RPE

ARPE-19 cells (L. M. Hjelmeland, ATCC #CRL-2302) were grown and maintained in DMEM-F12 medium supplemented with 10% FBS and incubated at 37° C. with a constant supply of 5% CO₂. ARPE-19 cells are spontaneously transformed human retinal pigment epithelial cells that conserve cellular biological and functional properties. All chemicals were purchased from Sigma Chemical Co. (St. Louis, Mo.) unless otherwise indicated.

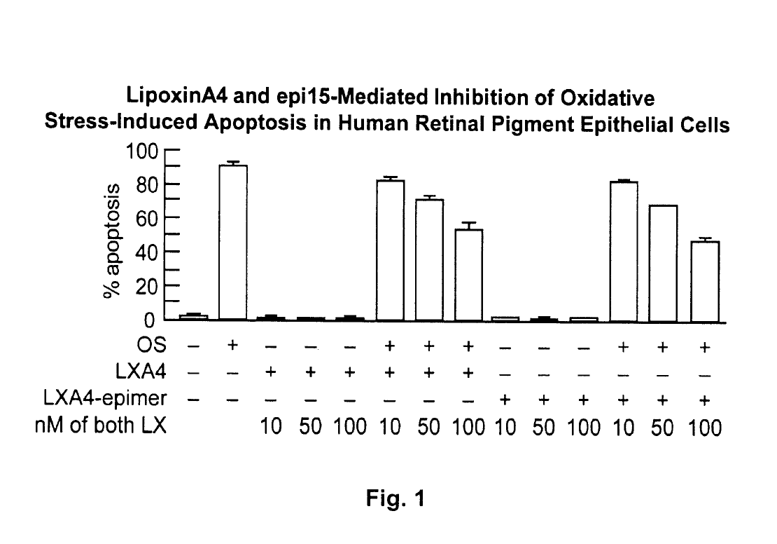

ARPE-19 cells growing in DMEM-F-12 medium for 72 h were serum starved for 8 h before induction of oxidative stress, as described in P. K. Mukherjee et al., “Photoreceptor outer segment phagocytosis attenuates oxidative stress-induced apoptosis with concomitant neuroportectin D1 synthesis,” PNAS, vol. 104, pp. 13158-13163 (2007). Oxidative stress was introduced by TNF-α (10 ng/ml) and H₂O₂ (600 uM) for 14 h and challenged with different concentrations of either lipoxin-A4 or lipoxin A4 epimer 15 (10 nM, 50 nM, and 100 nM; Calbiochem, Madison, Wis.), as indicated in FIG. 1. In initial experiments to test whether lipoxin caused stress, lipoxin A4 and lipoxin A4 epimer 15 were added to RPE cells in which oxidative stress had not been induced with TNF-α or H₂O₂. The apoptotic cell death was scored by Hoechst staining, as described in Mukherjee et al., 2007. The results were expressed as percentage inhibition of apoptosis by counting the Hoechst positive cells, and are shown in FIG. 1.

As shown in FIG. 1, the results indicate that the application of either lipoxin A4 or lipoxin A4 epimer 15 did not induce apoptosis in RPE cells. However, under inducement of oxidative stress, both lipoxin A4 and lipoxin A4 epimer 15 were able to inhibit apoptosis in RPE cells in a concentration-dependent manner. The highest inhibition was observed at 100 nM concentration of both lipoxin A4 and lipoxin A4 epimer 15. Negligible differences were seen between lipoxin A4 and lipoxin A4 epimer 15 in the ability to inhibit apoptosis, indicating that both lipoxin A4 and its analogs will be effective in inhibiting apoptosis.

### Example 2

### Lipoxins And NPD1 Effect On Apoptosis Inhibition Induced By Oxidative Stress In RPE

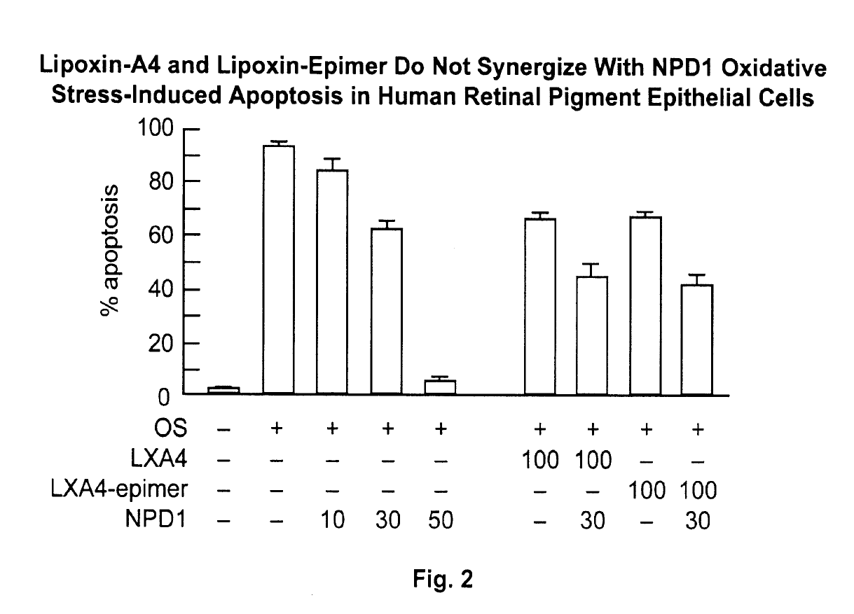

The growth of RPE cells, serum starvation, and induction of apoptosis were the same as described above in Example 1 and in Mukherjee et al., 2007. To test for a synergistic effect between lipoxin A4 and neuroprotectin 1 (NPD1), the oxidative-stressed RPE cells were treated with NPD1 (30 nM) and with lipoxin A4 or its analog (100 nM) for 14 h as indicated in FIG. 2. The percent apoptosis is expressed as described above in Example 1 as a percentage of the Hoechst positive cells.

As shown in FIG. 2, the results indicate that an additive effect of both lipoxins and NPD1 was found in this experiment. This indicates that the actions of NPD1 and lipoxins are mediated through different receptors and pathways. Thus, the effect of NPD1 when added with either lipoxin A4 or its analog is additive, and both would be useful in protecting RPE cells from apoptosis.

### Example 3

### Lipoxin A4 And Lipoxin A4 Epimer 15 Inhibited The Interleukin-1-β Induced COX-2-Promoter Construct In RPE

COX-2 is a proinflammatory protein that participates in RPE cell injury. ARPE-19 cells were grown over night in six well plates and then transfected with huCOX-2-LUC (−830) promoter construct (5 ug) for 24 h. Transfected cells were serum starved for 8 h before the addition of IL-1β (10 ng/ml). IL-1β treated cells were challenged with 100 nM, 500 nM, and 1000 nM concentrations of lipoxin A4 and lipoxin A4 epimer 15 for 14 h. Cells were then harvested, and luciferase assays were performed using luciferin as substrate.

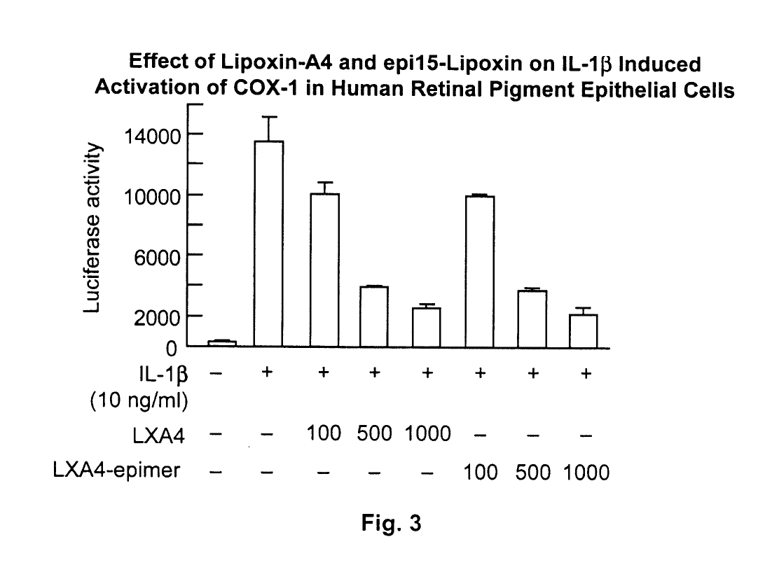

As shown in FIG. 3, the results indicate that both lipoxin A4 and lipoxin A4 epimer 15 inhibit IL-1β mediated induction of COX-2 promoter construct in RPE cells in a concentration-dependent manner. The lowest inhibition of 35% was observed at a concentration of 100 nM for both lipoxin A4 and lipoxin A4 epimer 15, and the highest was observed at 1000 nM.

Thus, Lipoxin A4 and its analogs protect human retinal pigment epithelial cells against oxidative stress-induced apoptosis. Lipoxin or its analogs, either alone or in combination with NPD1 (or its analogs), can be used to treat the dry form of AMD and other retinal degenerative diseases. The term “lipoxin A4 analogs” is understood to be compounds that are similar in structure to lipoxin A4 and that exhibit a biologically qualitatively similar effect as the unmodified lipoxin A4. The term includes stereochemical isomers of lipoxin A4, e.g., the aspirin-triggered 15-epimer lipoxin A4 (also, named lipoxin A4 epimer 15), and other known analogs, e.g., ATLa2 and the 3-oxa-lipoxin analogs (e.g., ZK-994 and ZK-142). See, U.S. Pat. Nos. 6,831,186 and 6,645,978; I. M. Fierro et al., “Lipoxin A4 and aspirin-triggered 15-epi-lipoxin A4 inhibit human neutrophil migration: Comparisons between synthetic 15 epimers in chemotaxis and transmigration with microvessel endothelial cells and epithelial cells,” Journal of Immunology, vol. 170, pp. 2688-2694 (2003); G. Bannenberg et al., “Lipoxins and novel 15-epi-lipoxin analogs display potent anti-inflammatory actions after oral administration,” Brit. J. Pharma. Vol. 143, pp. 43-52 (2004); and R. Scalia et al., “Lipoxin A4 stable analogs inhibit leukocyte rolling and adherence in the rat mesenteric microvasculature: role of P-selectin,” Proc. Natl. Acad. Sci. USA. vol. 94, pp. 9967-9972 (1997).

These compounds can be administered by methods known in the art, e.g., topical application or use of an implantable device, including a device that comprises a semipermeable membrane and retinal pigment epithelial cells genetically engineered to produce lipoxin A4, lipoxin A4 epimer 15, or an analog.

The term “effective amount” as used herein refers to an amount of lipoxin A4 or one of its analogs sufficient to protect a retinal pigment epithelial (RPE) cell from oxidative stress to a statistically significant degree (p<0.05). The term “effective amount” therefore includes, for example, an amount sufficient to prevent the degeneration of retinal pigment epithelial cells as found in diseases of the dry form of age-related macular degeneration or Stargardt's disease by at least 50%. The dosage ranges for the administration of lipoxin A4 or its analogs are those that produce the desired effect. Generally, the dosage will vary with the age and condition of the patient. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the degeneration of RPE cells by methods well known to those in the field, including as described in this application. Moreover, lipoxin A4 or its analogs can be applied in pharmaceutically acceptable carriers known in the art. The application can be oral, by injection, or topical.

Lipoxin A4 or its analogs may be administered to a patient by any suitable means, including orally, parenteral, subcutaneous, intrapulmonary, topically, and intranasal administration. Parenteral infusions include intramuscular, intravenous, intraarterial, or intraperitoneal administration. They may also be administered transdermally, for example in the form of a slow-release subcutaneous implant, or orally in the form of capsules, powders, or granules. The most preferred method will be topically or by an implant.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Lipoxin A4 or its analogs may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

Lipoxin A4 or its analogs may be formulated into therapeutic compositions as pharmaceutically acceptable salts. These salts include the acid addition salts formed with inorganic acids such as, for example, hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

Controlled delivery may be achieved by admixing the active ingredient with appropriate macromolecules, for example, polyesters, polyamino acids, polyvinyl pyrrolidone, ethylenevinylacetate, methylcellulose, carboxymethylcellulose, prolamine sulfate, or lactide/glycolide copolymers. The rate of release of lipoxin A4 or its analogs may be controlled by altering the concentration of the macromolecule.

Another method for controlling the duration of action comprises incorporating lipoxin A4 or its analogs into particles of a polymeric substance such as a polyester, peptide, hydrogel, polylactide/glycolide copolymer, or ethylenevinylacetate copolymers. Alternatively, lipoxin A4 or its analogs may be encapsulated in microcapsules prepared, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

In addition, lipoxin A4 or its analogs can be administered using an implantable device, similar to a contact lens with a semipermeable membrane to permit the diffusion of the active lipoxin compound. The implantable device could also carry a cell culture of retinal pigment epithelial cells that have been genetically engineered to produce lipoxin A4or its analogs.

The present invention provides a method of preventing, treating, or ameliorating degeneration of retinal pigment epithelial cells, comprising administering to a subject at risk for a disease or displaying symptoms for such disease, an effective amount of lipoxin A4 or an analog of lipoxin A4, e.g., lipoxin A4 epimer 15. The term “ameliorate” refers to a decrease or lessening of the symptoms or signs of the retinal degeneration being treated.

The complete disclosures of all references cited in this application are hereby incorporated by reference. In the event of an otherwise irreconcilable conflict, however, the present specification shall control.

1. A method for inhibition or prevention of degeneration of human retinal pigment epithelial cells, which comprises administering to a human an effective amount of one or more compounds selected from the group consisting of lipoxin A4 and lipoxin A4 analogs, such that degeneration of retinal pigment epithelial cells is inhibited or prevented.

2. A method for inhibition or prevention of dry form of age-related macular degeneration in a human, which comprises administering to a human an effective amount of one or more compounds selected from the group consisting of lipoxin A4 and lipoxin A4 analogs, such that the dry form of age-related macular degeneration is inhibited or prevented.

3. A method for inhibition or prevention of damage to human retinal pigment epithelial cells that would otherwise be caused by oxidative stress, which comprises administering to a human an effective amount of one or more compounds selected from the group consisting of lipoxin A4 and lipoxin A4 analogs, such that damage to retinal pigment epithelial cells is inhibited or prevented.

4. The method as in claim 1, additional comprising administration of an effective amount of a compound selected from the group consisting of docosahexaenoic acid and neuroprotectin D1.

5. The method as in claim 1, wherein the lipoxin A4 analog is lipoxin A4 epimer 15.

6. A medical composition for prevention of apoptosis of retinal pigment epithelial cells comprising one or more of a first compound selected from the group consisting of lipoxin A4 and lipoxin A4 analogs, and one or more of a second compound selected from the group consisting of docosahexaenoic acid and neuroprotectin D1.

7. The medical composition as in claim 6, wherein the lipoxin A4 analog is lipoxin A4 epimer 15.

8. An implantable cell culture device, the device comprising a semipermeable membrane permitting the diffusion of one or more of compounds selected from the group consisting of lipoxin A4 and lipoxin A4 analogs; and
containing genetically engineered retinal pigment epithelial cells to produce one or more of compounds selected from the group consisting of lipoxin A4 and lipoxin A4 analogs.

9. The implantable cell culture device of claim 8, wherein the lipoxin A4 analog is lipoxin A4 epimer 15.

10. A method for inhibiting retinal degeneration in a mammal, comprising implanting into the eye of the mammal an effective amount of the medical composition of claim 6 or the implantable cell culture device of claim 8.

11. The method for inhibiting retinal degeneration as in claim 10, wherein said retinal degeneration is associated with one or more diseases selected from the group consisting of age-related macular degeneration, retinitis pigmentosa, and glaucoma.

12. The method as in claim 10, additional comprising administration of an effective amount of one or more compounds selected from the group consisting of docosahexaenoic acid and neuroprotectin D1.

13. The method as in claim 2, additional comprising administration of an effective amount of a compound selected from the group consisting of docosahexaenoic acid and neuroprotectin D1.

14. The method as in claim 2, wherein the lipoxin A4 analog is lipoxin A4 epimer 15.

15. The method as in claim 3, additional comprising administration of an effective amount of a compound selected from the group consisting of docosahexaenoic acid and neuroprotectin D1.

16. The method as in claim 3, wherein the lipoxin A4 analog is lipoxin A4 epimer 15.
